(19) United States
(12) Patent Application Publication
(10) Pub. No.: US 2016/0256122 A1
(43) Pub. Date: Sep. 8, 2016

(54) Imaging System and Imaging Method

(71) Applicant: SIEMENS AKTIENGESELLSCHAFT, München (DE)

(72) Inventor: Oliver Heid, Erlangen (DE)

(73) Assignee: Siemens Aktiengesellschaft, Muenchen (DE)

(21) Appl. No.: 15/032,170

(22) PCT Filed: Oct. 22, 2014
(86) PCT No.: PCT/EP2014/072675

§ 371 Date: Apr. 26, 2016

(30) Foreign Application Priority Data
Oct. 28, 2013 (DE) 10 2013 221 818.4

(51) Int. Cl. A61B 6/00

(52) U.S. Cl. CPC A61B 6/4291; A61B 6/40

(57) ABSTRACT

An imaging system may include a radiation source for emitting radiation, a radiation detector having a regular arrangement of detector elements, and a shadow mask having a regularly repeating pattern. The shadow mask and the radiation detector may be arranged such that a projection of the pattern of the shadow mask is generated at the location of the detector by the radiation. A spatial repetition length of the projection of the pattern may differ from twice a spatial repetition length of the arrangement of the detector elements. Such imaging system may be used, for example, with the aid of the radiation detector, to measure the displacement of the projection of the pattern of the shadow mask at the location of the radiation detector which is effected by an object to be examined.

### CROSS-REFERENCE TO RELATED APPLICATIONS

This application is a U.S. National Stage Application of International Application No. PCT/EP2014/072675 filed Oct. 22, 2014, which designates the United States of America, and claims priority to DE Application No. 10 2013 221 818.4 filed Oct. 28, 2013, the contents of which are hereby incorporated by reference in their entirety.

### TECHNICAL FIELD

The present invention relates to an imaging system comprising a radiation source for emitting radiation, a radiation detector and a shadow mask. Furthermore, the invention relates to an imaging method using such a system. In particular, the invention relates to an imaging system with an x-ray source and an imaging method in which x-ray radiation is used.

### BACKGROUND

In known systems for x-ray imaging, there typically is measurement of the attenuation of the intensity of the x-ray radiation by the matter of an object to be examined, for example a human body part. Here, the absorption and scattering of the x-ray radiation in the object to be examined cause an attenuation of the radiation incident on the detector, said attenuation depending on the mass, the atomic number and the irradiated material volume. When a pixelated detector is used, images of the mass attenuation varying over different positions of the object are then obtained. Here, a two-dimensional image is obtained from one projection direction in the case of simple transillumination; by contrast, a three-dimensional data record of attenuation coefficients is reconstructed in computed tomography from a multiplicity of different transillumination measurements with different projection directions.

Strongly x-ray-absorbing tissue such as bones and calcification can be depicted very well by way of the known methods of absorption imaging. Achieving a high soft-tissue contrast in order to be able to image diagnostically relevant differences between various weakly absorbing tissue types well constitutes a greater challenge.

The method of phase contrast imaging using x-ray light was developed in recent years in order to improve the soft-tissue contrast in x-ray imaging. Within the scope of phase contrast x-raying, the spatially variable refractive index for x-ray radiation in an object is imaged instead of the absorption coefficients. To this end, the phase shift of the radiation waves after passing through the object is also measured in addition to the absorption. According to the prior art, a Talbot-Lau interferometer is used to this end, in which a plurality of very fine gratings are arranged partly upstream and partly downstream of the object to be examined and said gratings are displaced in a defined manner in relation to one another within the scope of a plurality of measurements carried out in succession. Good contrasts are obtained in weakly absorbing tissue structures using this method as the refractive index for x-ray radiation is determined much more strongly by the concentration of light atoms such as carbon, oxygen and nitrogen than the absorption coefficient. However, the known method requires much outlay in terms of equipment, mainly due to the production and positioning of the fine x-ray gratings, which require grating constants in the region of 2 μm.

### SUMMARY

One embodiment provides an imaging system comprising a radiation source for emitting radiation, a radiation detector with a regular arrangement of detector elements, and a shadow mask with a regularly repeating pattern, wherein the shadow mask and the radiation detector are arranged in such a way that a projection of the pattern of the shadow mask is generated by the radiation at the location of the detector, and wherein a spatial repetition length of the undistorted projection of the pattern deviates from twice a spatial repetition length of the arrangement of the detector elements.

In one embodiment, the spatial repetition length of the undistorted projection of the pattern deviates by at least 0.5% and at most 20% from twice the spatial repetition length of the arrangement of the detector elements.

In one embodiment, the spatial repetition length of the projection of the pattern and twice the spatial repetition length of the arrangement of the detector elements have an integer ratio, the integers of which lie between 1 and 100 in each case.

In one embodiment, the radiation source is an x-ray source and the radiation detector is an x-ray detector.

In one embodiment, an imaging region for positioning an object to be examined is arranged between the shadow mask and the radiation detector.

In one embodiment, the shadow mask has a two-dimensional regular pattern and the radiation detector has a two-dimensional regular arrangement of detector elements.

In one embodiment, the shadow mask has a regular alternation between regions absorbing the radiation of the radiation source weakly at best and strongly absorbing regions.

In one embodiment, the portion of at best weakly absorbing regions as part of an overall area of the shadow mask effective for the passage of radiation lies between 20% and 60%.

In one embodiment, the shadow mask has a material which comprises a metal and/or a metallic alloy.

In one embodiment, the regular pattern of the shadow mask is composed of rectangles.

In one embodiment, the regular pattern of the shadow mask has threefold and/or six-fold symmetry.

In one embodiment, the shadow mask has a regularly repeating pattern made of irregularly designed partial patterns.

Another embodiment provides an imaging method, wherein an imaging system as disclosed above is used to measure, with the aid of the radiation detector, a displacement of the projection of the pattern of the shadow mask at the location of the radiation detector caused by an object to be examined.

In one embodiment, a distribution of refractive indices of the object over different angular ranges of the projection is calculated from the distribution of the radiation intensities measured by the individual detector elements.

In one embodiment, the structure of the projection of the shadow mask is deconvolved from the measured intensity distribution when calculating the distribution of the refractive indices.

### BRIEF DESCRIPTION OF THE DRAWINGS

Example aspects and embodiments of the invention are described in detail below with reference to the drawings, in which:

FIG. 1 shows a schematic cross section of an imaging system according to a first exemplary embodiment, without an object to be examined,

FIG. 2 shows a schematic cross section of the same imaging system with an object to be examined,

FIG. 3 shows a section of the imaging system in the region of the radiation detector,

FIG. 4 shows a schematic illustration of the arising moiré pattern,

FIG. 5 shows a shadow mask according to a first exemplary embodiment,

FIG. 6 shows a shadow mask according to a second exemplary embodiment,

FIG. 7 shows a shadow mask according to a third exemplary embodiment, and

FIG. 8 shows a shadow mask according to a fourth exemplary embodiment.

### DETAILED DESCRIPTION

Embodiments of the invention provide an imaging system suitable for displaying the profile of the refractive indices in an object and which avoids the aforementioned disadvantages. Other embodiments provide an imaging method using such a system.

In some embodiments, the imaging system comprises a radiation source for emitting radiation, a radiation detector with a regular arrangement of detector elements, and a shadow mask with a regularly repeating pattern. The shadow mask and the radiation detector are arranged in such a way that a projection of the pattern of the shadow mask is generated by the radiation at the location of the detector. A spatial repetition length of the projection of the pattern deviates from twice a spatial repetition length of the arrangement of the detector elements.

Here, the respective spatial repetition length should be understood to mean the repetition length in respect of any predetermined spatial direction. Thus, it is sufficient if the repetition lengths deviate as specified in respect of at least one selected spatial direction.

The described imaging system may be particularly suited to imaging the spatial distribution of the refractive index, effective for the radiation, between the radiation source and radiation detector. Thus, the system can be used to image objects and materials in space by virtue of differences in the refractive indices being measured along different angular components of the radiation beam emitted by the source.

The shadow mask may be positioned between radiation source and radiation detector. In any case, it is positioned in such a way that a projection of the pattern is generated at the location of the detector by way of the shadow mask. In the case of a homogeneous refractive index for the radiation in the region between the radiation source and the detector, this projection is embodied in turn as a regular pattern at the location of the radiation detector. Depending on the type, and possibly on the focusing, of the radiation profile, the projection can be magnified, reduced or have the same size as the size of the shadow mask. When an x-ray source is used as a radiation source with a focal spot that is as small as possible as an emanating point for the radiation, this advantageously is a magnified projection as it is not easy to focus x-ray radiation by optical elements.

If the spatial repetition length of such an undistorted projection exactly equals the spatial repetition length of the arrangement of detector elements, a substantially uniform intensity profile would be measured with the aid of this radiation detector. A deviation of the intensities measured by the various detector elements then would be caused substantially only by possibly present different spacings between the detector elements and the radiation source and/or by possibly present absorbing materials in different regions of the radiation beam. However, the shadow mask would not lead to different intensities on the individual detector elements in the case of such an adjustment of the spatial repetition lengths.

In the case of an exact adjustment of the spatial repetition length of the undistorted projection to twice the repetition length or a different integer multiple n of the repetition length of the detector arrangement, a substantially uniform intensity profile would emerge for each pair or each group of n detector elements. Then, the pattern of the shadow mask would be imaged within the pair or the group of n detector elements. By way of example, a shadow mask with alternating strips of equal length with in each case strong and weak absorption would lead to alternating weak and strong measurement signals of the adjacent detector elements.

However, the repetition length of the undistorted projection is precisely not adjusted to twice the repetition length of the detector arrangement in the disclosed imaging system. Expediently, it is not adjusted to the single repetition length of the detector arrangement either. Expediently, it only deviates slightly from an integer multiple of the repetition length of the detector arrangement, for example by up to 20% there above or there below.

This slight maladjustment leads to a beat effect in the profile of the intensities measured by the individual detector elements. As a result of the moiré effect, a pattern superposed on the finer pattern of the projection of the shadow mask is created, the spatial repetition length of which is significantly greater than the repetition lengths of the projection of the shadow mask and the detector arrangement. The closer the repetition lengths of projection and the detector arrangement lie next to one another, i.e. the smaller the maladjustment is, the greater the repetition length of the superposed moiré pattern is.

With the disclosed imaging system, the superposed moiré pattern reacts sensitively to small disturbances in the interaction of the two underlying periodic structures. In particular, slight distortions in the pattern of the projection of the shadow mask lead to spatially substantially larger distortions in the superordinate moiré pattern. However, a slight distortion in the pattern of the projection of the shadow mask is caused precisely by a spatially varying refractive index of an object or material positioned in the beam path. Thus, the proposed imaging system exploits the effect that an originally uniform angular distribution of the radiation is distorted into an irregular angular distribution by a variation of the refractive index in the region of the beam. This in turn brings about a distortion of the projection of the pattern of the shadow mask and, as a result thereof, a magnified distortion in the superposed moiré pattern. As a result of this magnification of the distortion, the measurement of very small angle changes is simplified. The extent of the magnification is determined by the precise selection of the maladjustment of the two underlying regular structures. As a result of this, there can be a suitable selection of the parameters for each imaging situation, in which the magnification is adjusted to the required sensitivity and to the required spatial resolution possible in terms of equipment.

Some embodiments provide an imaging method that uses an imaging system as disclosed herein to measure, with the aid of the radiation detector, a displacement of the projection of the pattern of the shadow mask at the location of the radiation detector caused by an object to be examined. Some embodiments of the imaging system may additionally include one or more of the following features:

The spatial repetition length of the undistorted projection of the pattern can advantageously deviate by at least 0.5% and at most 20% from twice the spatial repetition length of the arrangement of the detector elements. As a result of a maladjustment in this range, the created superposed moiré pattern is magnified by a factor of between approximately 200 and approximately 5 in relation to the two finer patterns. This advantageously enables an increased sensitivity when measuring small refractive index differences in the passed-through volume.

Particularly in the case of magnifications between 5 and 20, it is still possible to achieve relatively high spatial accuracy when determining the refractive index differences despite the loss, as a matter of principle, of spatial resolution.

In some embodiments, spatial repetition length of the undistorted projection of the pattern deviates by at least 1% and at most 10% from twice the spatial repetition length of the detector arrangement. Then, the magnification factor advantageously lies between approximately 100 and approximately 10.

The spatial repetition length of the projection of the pattern and twice the spatial repetition length of the arrangement of the detector elements can have an integer ratio, the integers of which lie between 1 and 100, particularly advantageously between 1 and 20, in each case. The selection of such an integer ratio is advantageous in that the beat pattern created on the detector elements in the case of an undistorted projection in each case repeats in a regular manner after a group of detector elements next to one another. This can additionally simplify an evaluation of a disturbance of this ideal beat pattern by an object to be examined.

The radiation source can be an x-ray source and the radiation detector can be an x-ray detector. The proposed arrangement is particularly suitable for an imaging system based on x-ray radiation since the variations in the refractive indices in most objects are very small precisely for x-ray radiation. By exploiting the moiré effect, it is possible to increase the sensitivity of the imaging system in respect of such small variations to such an extent that it is also possible to make visible the refractive index differences for x-ray radiation. In x-ray imaging, the measurement of such very small displacements as a result of refractive index differences is additionally made more difficult by virtue of most x-ray detectors only having a low spatial resolution. By way of example, the detector elements of the x-ray detector can have dimensions of between 10 μm and 1000 μm. Particularly in the lower part of this range, i.e., for example, in the case of a repetition length of the detector elements between 10 μm and 200 μm, the spatial crosstalk between adjacent detector elements is very large in the case of x-ray detectors. This is true both for x-ray detectors based on scintillators and for x-ray detectors operating according to the principle of the direct conversion of x-ray radiation, for example in a semiconductor material. The spatial crosstalk between adjacent detector elements can still be significant even in the case of a repetition length between 200 μm and several millimeters. This effect leads to the achievable spatial resolution, precisely in x-ray detectors, being substantially coarser than would be suggested by the spatial repetition length. It is also precisely for this reason that embodiments of the invention may be particularly suitable to make visible refractive index differences for x-ray radiation because, as a result of the magnification effect, the superordinate moiré pattern can be set to be so large that spatial crosstalk of the detector pixels no longer plays a significant role on the resulting scale.

An imaging region for positioning an object to be examined can be arranged between the shadow mask and the radiation detector. In principle, the object to be examined can be arranged at any position between radiation source and detector in order to influence, by way of the varying refractive index thereof, the projection of the pattern of the shadow mask at the location of the detector. However, it is advantageous, precisely in the case of an x-ray system for medical imaging, if the object, i.e. usually the patient in this case, is arranged between the shadow mask and the radiation detector. This is because the dose of the radiation incident on the object is already reduced by the shadow mask to the image-effective portion of the radiation and the exposure of the patient to damaging x-ray radiation is lower.

The shadow mask can have a two-dimensional regular pattern and the radiation detector can have a two-dimensional regular arrangement of detector elements. Expediently, these two two-dimensional arrangements can lie substantially perpendicular to a mean beam propagation direction. Particularly advantageously, the repetition lengths of projection pattern and detector arrangement then can deviate from one another in the specified manner in a plurality of spatial directions such that the generation of a two-dimensional moiré pattern allows the determination of refractive index variations in two dimensions. In other words, this can be used to obtain a two-dimensional image of refractive index variations across the mean radiation propagation direction. The repetition lengths of detector and/or shadow mask can be different in different spatial directions of the image plane. Alternatively, they can also have the same embodiment in different spatial directions.

The shadow mask can have a regular alternation between regions absorbing the radiation of the radiation source weakly at best and strongly absorbing regions. By way of example, regions absorbing weakly at best should be understood to mean regions in which the absorption of the radiation is at most 20%, particularly advantageously at most 10%. In any case, the absorption in these regions can be greater than zero, without this impairing the basic functionality of the imaging system. It is only advantageously as low as possible so that the source need not produce unnecessarily high and unused radiation intensities. The at best weakly absorbing regions can also be embodied as gaps in the shadow mask which therefore do not have any absorption in this case. However, in the case of x-ray radiation, they can also be made from light, weakly absorbing material or, in the case of visible radiation, they can be embodied as optically transparent regions. By way of example, strongly absorbing regions should be understood to mean regions in which the absorption of the radiation is at least 50%, particularly advantageously at least 75%. In any case, there need not be complete absorption of the radiation in these regions. Particularly in the case of x-ray imaging, it is difficult to produce fine shadow masks which have a precisely formed structure and virtually completely absorb the radiation in the absorption regions due to the low attenuation coefficients of many materials. However, it is sufficient for the proposed system if there is a sufficiently high modulation of the radiation passing through the shadow mask in order to be imaged as a pattern or as a beat pattern on a given detector. The boundary between the strongly absorbing and weakly absorbing regions need not be a sharp edge; there can also be a transition region therebetween with mean absorption values.

The portion of at best weakly absorbing regions as part of an overall area of the shadow mask effective for the passage of radiation can advantageously lie between 20% and 60%, particularly advantageously between 25% and 50%. By way of example, the effective overall area of the shadow mask is arranged in a plane perpendicular to the mean beam direction. By way of example, the portion can be approximately 50%; in particular, the at best weakly absorbing and strongly absorbing regions can have equal size and occur with equal frequency. However, this is not necessary. In particular, an even sharper pattern can, as it were, be generated with lower portions of at best weakly absorbing regions, which pattern generates even tighter regions with high radiation intensity in the projection on the detector in the case of the same spatial frequency. This can contribute to an improved representation of the superordinate moiré pattern and hence of the refractive index variations.

The shadow mask can have a material which comprises a metal and/or a metallic alloy. The use of masks containing metals is advantageous, in particular for x-ray imaging, since metals have a high attenuation coefficient for x-ray radiation. Particularly advantageously, the shadow mask can contain gold, lead and/or tungsten. Advantageously, it can have a thickness of at least 100 μm, particularly advantageously at least 200 μm in the beam direction. When using radiation in the visible range, use can advantageously be made of optically opaque materials, particularly advantageously black materials.

The regular pattern of the shadow mask can be composed of rectangles. By way of example, the pattern can have a substantially checkerboard-like embodiment. However, it can also be embodied as a two-dimensional alternating pattern of strongly absorbing and at best weakly absorbing rectangles, in which, for example, the at best weakly absorbing rectangles are slightly smaller than the distance therebetween. Alternatively, there can also be a regular arrangement of squares and/or rectangles, in which, for example, only each fourth quadrant is transmissive or weakly absorbing. By way of example, such a pattern of the shadow mask can be obtained effectively by switching two mutually perpendicular strip masks in succession.

The regular pattern of the shadow mask can have threefold and/or six-fold symmetry. By way of example, the pattern of the shadow mask can have hexagonal symmetry. Such a honeycomb-like pattern is particularly suitable for manufacturing regular shadow masks with a high accuracy and high x-ray absorption in a simple manner. By way of example, such honeycomb-like masks can be produced by rolling and stamping lead sheets.

The shadow mask can also be generated from a solid body by removing material at regular intervals. By way of example, circular cylindrical holes or holes with a rectangular or hexagonal footprint can be generated. By way of example, the holes can be generated by etching. Alternatively, the regular structure of the shadow mask can also be produced by three-dimensional printing methods, stereo lithography, laser sintering and/or injection molding methods. Thus, provision can be made of cylindrical recesses. The base area of the cylinders can have, for example, a rectangular, hexagonal or circular form.

The shadow mask can have a regularly repeating pattern made of irregularly designed partial patterns. Here, irregular should at least be understood to mean that the partial pattern does not have the same symmetry as the regular overall pattern. It can be a pattern entirely without symmetry or it can have a different symmetry than the superordinate pattern. By way of example, it can be a complex partial pattern which, as a type of stamp, repeats again and again and thus impresses a characteristic signature onto the moiré pattern measured by the detector. The advantage of this embodiment is that the resulting image contains a high portion of low spatial frequencies and that relatively small variations in the refractive index can be detected particularly sensitively. However, to this end the mask function needs to be deconvolved from the measured image within the scope of mathematical post processing.

In some embodiments, a distribution of refractive indices of the object over different angular ranges of the projection can be calculated in the method from the distribution of the radiation intensities measured by the individual detector elements. To this end, it is possible, for example, to carry out a comparison of the images of the shadow mask measured with and without object. Differences between these various images can be formed for calculating the refractive index distribution. Alternatively or additionally, it is possible to calculate Fourier transforms of the measured images. Then, the images can be, for example, compared to one another and/or combined by calculation in the spatial frequency space. They can be convolved and/or deconvolved with other functions. The spectra obtained in the spatial frequency space can be transformed back into the image space after processing. In addition to the distribution of the refractive indices of the object, the distribution of the absorption coefficients can also be determined.

The structure of the shadow mask and/or the projection of the shadow mask can advantageously be deconvolved from the measured intensity distribution when calculating the distribution of the refractive indices. This is expedient, in particular, if the shadow mask has a regularly repeating pattern made of partial patterns with an irregular embodiment. In this manner, the signature of this partial pattern impressed on the moiré pattern can be removed from the image by calculation.

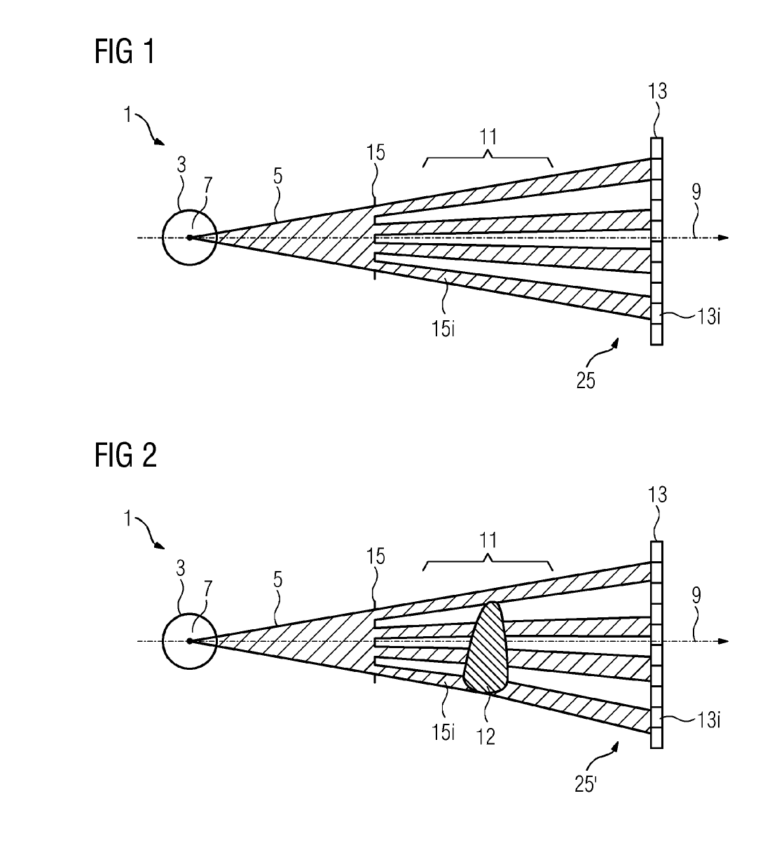

FIG. 1 shows a schematic cross section of an imaging system 1 according to an example embodiment of the invention. What is shown is the beam path of the radiation 5 from the radiation source 3, through the shadow mask 15 and the imaging region 11 and to the radiation detector 13. In this example, the radiation source 5 is an x-ray source with a small focal spot 7, which forms a virtually punctiform initial point for the x-ray radiation 5 emanating from the source 5 in a conical manner. In this case, the shadow mask 15 is a plane mask which extends substantially perpendicular to the mean radiation direction 9. The shadow mask 15 has a regular two-dimensional pattern. In the sectional plane shown in FIG. 1, transmissive regions alternate periodically with strongly absorbing regions. A plurality of radiation beams 15i pass through the transmissive regions, of which only four are shown in the figure in an exemplary manner. However, in reality, the shadow mask 15 has a multiplicity of transmissive regions in each spatial direction, and so a multiplicity of radiation beams 15i are also formed. By way of example, several thousand of such radiation beams 15i can lie in such an exemplary sectional plane. The radiation beams continue to propagate after the shadow mask and they are incident on the radiation detector 13 after passing through the imaging region 11. FIG. 1 depicts the case of a reference measurement without an object 12 to be imaged. In this case, a regular undistorted projection 25, which represents a magnified image of the pattern of the shadow mask 15 in this example, arises in the plane of the radiation detector 13. The radiation detector 13 has a plurality of detector elements 13i, with these in this case also only being reproduced by a small number in an exemplary manner. In real embodiment examples, the detector 13 will have a multiplicity of such detector elements 13i, for example also several thousand in each spatial direction of the detector plane in this case. The pitch of the detector elements 13i is similar but not exactly equal to half the pitch of the shadowed and irradiated regions of the projection 25 such that a beat pattern arises in the intensity distribution measured by the detector elements. However, a greater number than the number of radiation beams 15i and detector elements 13i shown in the schematic illustration is required for such a beat pattern.

FIG. 2 shows a corresponding schematic cross section of the same imaging system 1 with an object 12 to be examined in the imaging region 11. Additionally, the imaging system 1 can comprise an arrangement (not shown here) for holding an object 12 to be examined. By way of example, this can be a patient couch or an apparatus for receiving a body part. For applications in mammography, the holder typically includes two plates for fixing and compressing the female chest.

When passing through the object 12 to be examined, the x-ray radiation 5 is both attenuated in terms of its amplitude by absorption and distorted in the direction of the individual radiation beams 15i by refractive index differences. This distortion causes a slight distortion in the pattern of the projection 25′ incident on the radiation detector 13. This distortion of the projection 25′ can be made visible more easily by the two not quite corresponding gratings of the projected pattern 25′ and the detector arrangement 13 and by the arising beat effects.

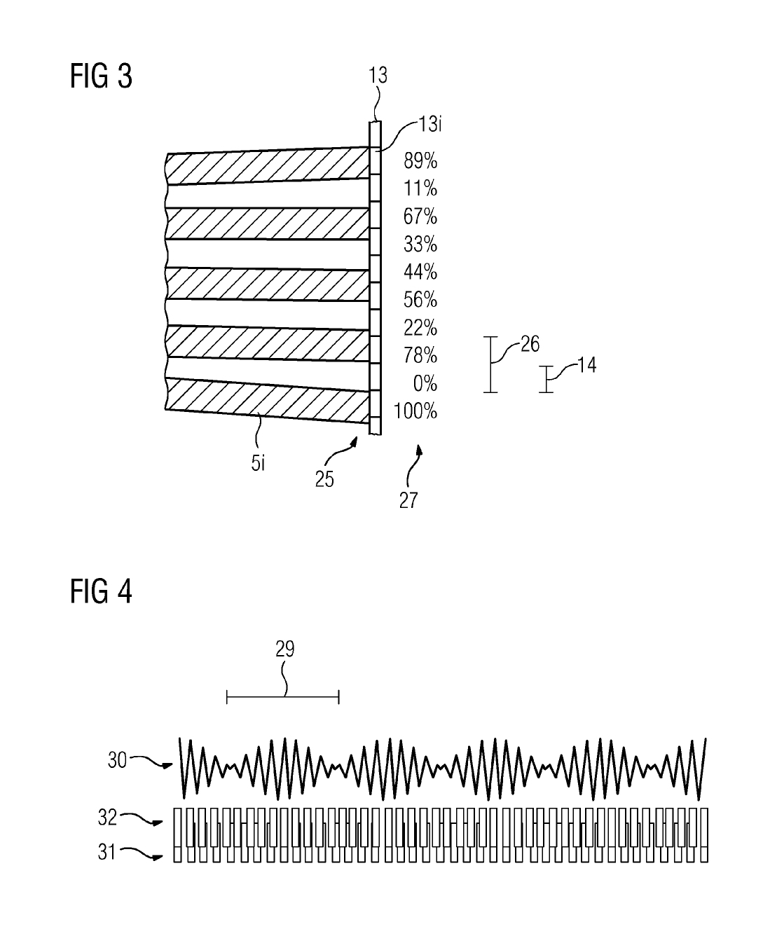

FIG. 3 shows a section of the imaging system 1 in the region in which the radiation beams 15i are incident on the radiation detector 13. No object 12 is positioned in the beam path in this example and the radiation beams image an undistorted projection 25 on the detector 13. The slight maladjustment of the two gratings is embodied in such a way in this example that the spatial repetition length 26 of the undistorted projection 25 is approximately 10% greater than twice the spatial repetition length 14 of the detector arrangement 13. The distribution of relative intensities specified to the right of the individual detector elements 13i arises as a result of this slight maladjustment. This intensity pattern is generated as a moiré pattern or spatial beat pattern from the two gratings with a similar size scale not adjusted to one another. An introduction of an object 12 into the beam path now brings about a characteristic distortion of this beat pattern. FIG. 4 shows the embodiment of the beat pattern 30 more clearly for a larger number of detector elements 13i. The grating of the projection 31 is reproduced schematically here as a regular bar grating made of light and dark regions. The grating of the detector 32 is also reproduced here in a very simplified manner as a regular bar grating, wherein each bar and each gap between two bars should be understood to be a detector element 13i in this case. In this partly overlapping bar representation, the embodiment of a beat pattern can already be easily identified by eye. In the curve placed there over, the arising moiré pattern 30 is reproduced as a schematic intensity distribution which is superposed on the profile of the intensities measured by the individual detector elements 13i. Here, the repetition length 29 of the beat pattern 30 is a multiple of the repetition lengths of the two underlying gratings 31 and 32. Expressed in spatial frequencies, the spatial frequency of the beat emerges as magnitude of the difference between the spatial frequencies of the two underlying smaller gratings 31 and 32. Here, the spatial frequency in each case is the inverse of the respective spatial repetition length 14, 16 or 29.

A slight distortion in the pattern of the projection 25′, which is measured with an object 12 situated in the beam path, will now be made visible particularly sensitively by way of the moiré effect. The very small refractive index differences, which are present in most objects 12 in the range of x-ray radiation, can be made visible particularly well by the distortion of the magnified beat pattern. However, this higher sensitivity is paid for by a lower spatial resolution since the spatial resolution for the measured refractive index differences corresponds approximately to the repetition length 29 of the beat pattern 30. Already from this, it is clear that the numbers of radiation beams 15i and detector elements 13i shown in FIGS. 1 and 2 should be understood to be only representative for substantially larger numbers of elements.

FIG. 5 shows a first preferred exemplary embodiment for a shadow mask 15, which finds use in the imaging system according to FIG. 1. What is shown is an exemplary portion of a spatially extended regular arrangement. In this example, the shadow mask 15 is embodied as a checkerboard-like pattern made of strongly absorbing regions 15b and weakly absorbing regions 15a. The weakly absorbing regions 15a can also be understood to be non-absorbing regions. That is to say they can be embodied as regions completely free from material, through which the radiation 5 can pass without impediment. The strongly absorbing regions 15b are embodied as strongly x-ray-absorbent material for the imaging system 1 from FIG. 1; by way of example, they can contain gold, tungsten and/or lead. The layer thickness of the shadow mask in the radiation direction is selected to be so high that, for example, at least 50% of the radiation is absorbed. The manufacturing and/or the mechanical stability of such a checkerboard-like shadow mask can be improved if the weakly absorbing regions 15a are not completely empty but if they are at least partly filled by a weakly absorbing material.

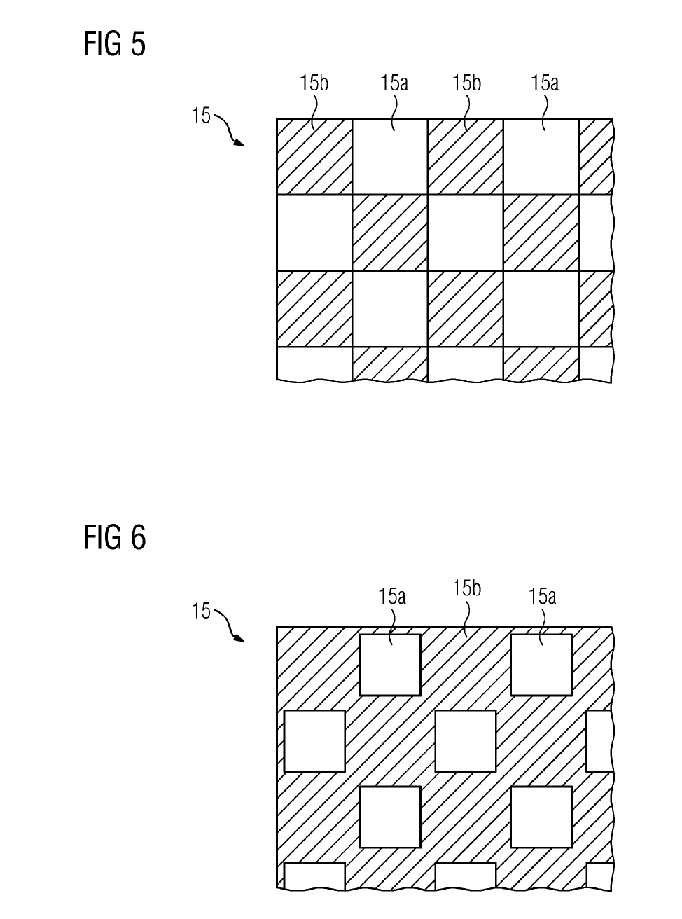

FIG. 6 shows a second preferred exemplary embodiment for a shadow mask 15. In this case too, the alternation of the strongly absorbing regions 15b and the weakly absorbing regions 15a is a checkerboard-like arrangement of rectangles. However, the weakly absorbing regions 15a have a smaller embodiment in this example, and so, overall, cover less than 50% of the area effective for the radiation passage. This can simplify the production and/or mechanical stability of a shadow mask in which no matter at all is present in the weakly absorbing regions 15a.

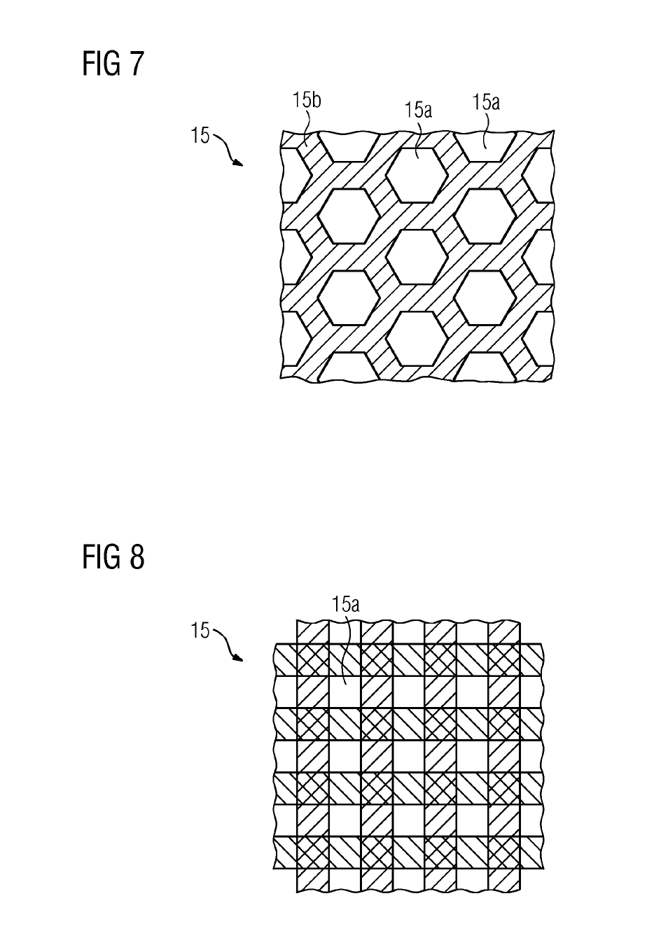

FIG. 7 shows an alternative exemplary embodiment of a shadow mask 15, in which a multiplicity of six-sided holes 15a are arranged in a hexagonal pattern. By way of example, such an arrangement can be obtained relatively easily from a stack made of formed lead sheets. Alternatively, a solid body made of a strongly absorbing material can also be provided with holes. By way of example, these can also be circular cylindrical holes.

FIG. 8 shows a portion of a shadow mask 15 according to a fourth exemplary embodiment. In this example, the shadow mask 15 is formed by an arrangement, switched in series in the radiation direction 9, made of two mutually perpendicular, respectively one-dimensional line gratings. By way of example, such one-dimensional line gratings can be produced in a simple manner as a stack of metal plates arranged next to one another. Here, the metal plates each can be held by a support made of material which weakly absorbs radiation. If a shadowing portion of 50% is selected for each one-dimensional grating, a shadowing portion of 75% emerges for the superposed grating. However, the underlying one-dimensional gratings can alternatively also be selected to be more tight or more transmissive in order to obtain a predetermined transmissivity of the superordinate grating.

By way of example, when use is made of x-ray radiation, the shadow mask 15 can have a thickness of between 100 μm and several millimeters in order to absorb the majority of the high-energy radiation 5. The weakly absorbing regions 15b can then be embodied, for example, as a family of recesses extending parallel to one another in a strongly absorbing body. Alternatively, they can also be embodied as a family of slightly angled recesses which, overall, are aligned to the focus of the radiation 5, that is to say, for example, on the focal spot 7 of the x-ray source.

What is claimed is:

1. An imaging system comprising:
a radiation source configured to emit radiation,
a radiation detector including a regular arrangement of detector elements, and
a shadow mask with a regularly repeating pattern,
wherein the shadow mask and the radiation detector are arranged such that a projection of the pattern of the shadow mask is generated by the radiation at a location of the detector, and
wherein a spatial repetition length of the undistorted projection of the pattern deviates from twice a spatial repetition length of the arrangement of the detector elements.

2. The imaging system of claim 1, wherein the spatial repetition length of the undistorted projection of the pattern deviates by at least 0.5% and at most 20% from twice the spatial repetition length of the arrangement of the detector elements.

3. The imaging system of claim 1, wherein the spatial repetition length of the projection of the pattern and twice the spatial repetition length of the arrangement of the detector elements have an integer ratio based on integers in the range of 1-100.

4. The imaging system of claim 1, wherein the radiation source is an x-ray source and the radiation detector is an x-ray detector.

5. The imaging system of claim 1, comprising an imaging region for positioning an object to be examined, the imaging region is arranged between the shadow mask and the radiation detector.

6. The imaging system of claim 1, wherein the shadow mask has a two-dimensional regular pattern, and the radiation detector has a two-dimensional regular arrangement of detector elements.

7. The imaging system of claim 1, wherein the shadow mask has a regular alternation between regions absorbing the radiation of the radiation source weakly at best and strongly absorbing regions.

8. The imaging system of claim 7, wherein the portion of at best weakly absorbing regions as part of an overall area of the shadow mask effective for the passage of radiation lies between 20% and 60%.

9. The imaging system of claim 1, wherein the shadow mask has a material comprising at least one of a metal allow or a metallic alloy.

10. The imaging system of claim 1, wherein the regular pattern of the shadow mask is composed of rectangles.

11. The imaging system of claim 1, wherein the regular pattern of the shadow mask has at least one of a threefold symmetry or a six-fold symmetry.

12. The imaging system of claim 1, wherein the shadow mask has a regularly repeating pattern made of irregularly designed partial patterns.

13. An imaging method, comprising:
providing an imaging system including a radiation source configured to emit radiation, a radiation detector including a regular arrangement of detector elements, and a shadow mask with a regularly repeating pattern, wherein the shadow mask and the radiation detector are arranged such that a projection of the pattern of the shadow mask is generated by the radiation at a location of the detector, and wherein a spatial repetition length of the undistorted projection of the pattern deviates from twice a spatial repetition length of the arrangement of the detector elements, and
using the imaging system to measure, via the radiation detector, a displacement of the projection of the pattern of the shadow mask at the location of the radiation detector caused by an object to be examined.

14. The imaging method of claim 13, comprising:
calculating a distribution of radiation intensities measured by the individual detector elements of the radiation detector, and
calculating a distribution of refractive indices of the object over different angular ranges of the projection from the calculated distribution of measured radiation intensities.

15. The imaging method of claim 14, wherein calculating the distribution of the refractive indices includes deconvolving a structure of the projection of the shadow mask from the measured intensity distribution.
